(19) United States
(12) Patent Application Publication
(10) Pub. No.: US 2026/0216212 A1
(43) Pub. Date: Jul. 30, 2026

(54) USE OF MEROTERPENOID I IN PREPARATION OF ANTI-HEART FAILURE DRUG

(71) Applicant: GUANGZHOU UNIVERSITY OF CHINESE MEDICINE(GUANGZHOU INSTITUTE OF CHINESE MEDICINE), Guangdong (CN)

(72) Inventors: Zhongqiu LIU, Guangdong (CN); Hui CUI, Guangdong (CN); Yuanyuan CHENG, Guangdong (CN)

(73) Assignee: GUANGZHOU UNIVERSITY OF CHINESE MEDICINE(GUANGZHOU INSTITUTE OF CHINESE MEDICINE), Guangdong (CN)

(21) Appl. No.: 19/630,458

(22) Filed: Mar. 27, 2026

Related U.S. Application Data
(63) Continuation of application No. PCT/CN2025/137146, filed on Nov. 24, 2025.

(30) Foreign Application Priority Data
Oct. 22, 2024 (CN) 202411473519.1

(51) Int. Cl. A61K 31/585; A61P 9/04

(52) U.S. Cl. CPC A61K 31/585; A61P 9/04

(57) ABSTRACT

Disclosed in the present invention is use of meroterpenoid I in preparation of an anti-heart failure drug. In the present invention, mouse heart failure models induced by doxorubicin or left anterior descending coronary artery ligation are treated with meroterpenoid I isolated and purified from marine fungus Aspergillus terreus GZU-311. It is found that the meroterpenoid I can improve cardiac function in mice, reduce cardiomyocyte injury, inhibit myocardial connective tissue thickening, ameliorate disordered arrangement of cardiomyocytes, and reduce inflammatory cell infiltration, thereby exhibiting an anti-heart failure effect. Therefore, the present invention provides the use of meroterpenoid I in the preparation of an anti-heart failure drug. The present invention enriches pharmaceutical molecules available for the treatment of heart failure, thereby facilitating the development of anti-heart failure drugs and their clinical treatment.

### CROSS-REFERENCE TO RELATED APPLICATION

This application is a continuation of PCT application No. PCT/CN2025/137146, filed on Nov. 24, 2025, which claims priority right of Chinese patent application No. 202411473519.1 filed on Oct. 22, 2024. The entirety of each of the above-mentioned patent applications is hereby incorporated by reference herein and made a part of this specification.

### BACKGROUND

### Technical Field

The present invention belongs to the technical field of biopharmaceuticals, and specifically relates to use of meroterpenoid I in preparation of an anti-heart failure drug.

### Description of Related Art

Heart failure, abbreviated as HF, refers to a pathophysiological state and clinical syndrome characterized by a reduced cardiac pumping function, failing to adequately eject the venous return blood to meet the metabolic demands of the full-body tissues. It has the characteristics of a high re-hospitalization rate, a high disability rate, a high mortality rate, and the like.

Currently, the anti-heart failure drugs clinically commonly used primarily include renin-angiotensin system inhibitors, anti-aldosterone drugs, diuretics, beta-blockers, and cardiac glycosides. Although the aforementioned drugs have a certain anti-heart failure effect clinically, their long-term use can lead to drug tolerance, as well as a series of side effects and adverse reactions, such as orthostatic hypotension, depression, elevated blood lipids, hypoglycemia, peripheral circulatory disturbance, fatigue, tracheospasm, suppressing myocardial contractility, and conduction block. Therefore, there is an urgent need to find new pharmaceutical molecules to replace existing clinical drugs, in order to overcome patient tolerance to existing anti-heart failure drugs and the adverse reactions they cause.

The secondary metabolites of marine fungi constitute a vital component of natural products, and have the characteristics of sustainability, environmental friendliness, rich and diverse metabolites, small side effects, and the like. They are a crucial source for the screening of natural drugs. Meanwhile, the secondary metabolites of marine fungi have a wide range of physiological activities, such as antibacterial, antitumor, antifibrotic, anti-Alzheimer's disease, anti-heart failure, and enzyme-inhibitory effects, thereby injecting new momentum into the innovation of modern drugs and the research and development of innovative drugs. Currently, the search for new pharmaceutical molecules from marine fungi has become a research hotspot both domestically and internationally.

Although the development of cardiovascular disease is closely associated with inflammation, cardiomyocyte fibrosis, and the like, not just any compound having anti-inflammatory or antifibrotic effects can be utilized for the treatment of heart failure. For example, drugs prepared from thiazolidinedione compounds can inhibit renal inflammatory responses, inhibit renal cell fibrosis, and improve renal microcirculation (DOI: 10.3969/j.issn.1672-6170.2009.01.044). However, this class of drugs may lead to water and sodium retention and fluid overload, thereby inducing or exacerbating heart failure, and increasing the risk of mortality from heart failure or cardiovascular disease. Although the secondary metabolites of marine fungi are abundant, obtaining compounds with anti-heart failure effects from them is no easy task.

### SUMMARY

Aiming at the aforementioned problems of the prior art, the present invention provides a meroterpenoid, namely the meroterpenoid I of the present invention, capable of being used for preparing an anti-heart failure drug.

A first objective of the present invention is to provide use of meroterpenoid I in preparation of an anti-heart failure drug.

A second objective of the present invention is to provide an anti-heart failure drug.

The above objectives of the present invention are implemented through the following technical solutions:

In the present invention, mouse heart failure models induced by doxorubicin, left anterior descending coronary artery ligation, or angiotensin II are treated with meroterpenoid I isolated from the fermentation culture of marine fungus Aspergillus terreus GZU-311. It is found that the meroterpenoid I has an anti-heart failure effect. Therefore, the present invention claims the use of the meroterpenoid I in the preparation of an anti-heart failure drug.

Specifically, the structural formula of the meroterpenoid I is shown as formula (I):

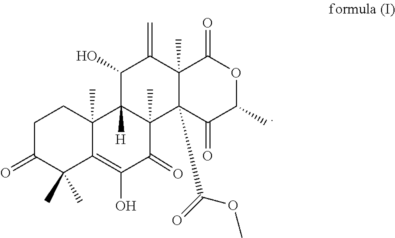

Specifically, the meroterpenoid I is capable of improving cardiac function.

Specifically, the improving cardiac function refers to that the meroterpenoid I is capable of increasing a cardiac ejection fraction and a fractional shortening.

Specifically, the meroterpenoid I is capable of reducing the angiotensin II-induced elevation of systolic and diastolic blood pressures.

Specifically, the meroterpenoid I is capable of reducing cardiomyocyte injury.

Specifically, the meroterpenoid I is capable of inhibiting myocardial connective tissue thickening.

Specifically, the meroterpenoid I is capable of ameliorating disordered arrangement of cardiomyocytes.

Specifically, the meroterpenoid I is capable of reducing inflammatory cell infiltration.

Specifically, the heart failure is chronic heart failure or ischemic heart failure.

Specifically, the heart failure is doxorubicin-induced heart failure.

Specifically, the heart failure is heart failure with preserved ejection fraction.

Specifically, the meroterpenoid I is isolated and purified from a fermentation culture of marine fungus Aspergillus terreus GZU-311.

Specifically, a medium used for fermentation cultivation is a solid maize medium formulated from maize and seawater.

Specifically, a method for preparing the meroterpenoid I includes the following steps:
S1. preparing a seed solution of marine fungus Aspergillus terreus GZU-311;
S2. inoculating the seed solution prepared in S1 into a solid maize medium for cultivation to obtain a fermentation culture;
S3. extracting the fermentation culture obtained from the cultivation in S2 two to five times with methanol, concentrating extracts, and extracting a resulting concentrated extract with ethyl acetate to obtain a crude ethyl acetate extract; and
S4. isolating the crude ethyl acetate extract obtained in S3 using normal-phase silica gel chromatography, and then conducting gradient elution in sequence using petroleum ether/ethyl acetate mixed solutions with petroleum ether volume fractions of 10%, 20%, 30%, 40%, 50%, 60%, and 70%, respectively; collecting a 40% petroleum ether/ethyl acetate eluate fraction, and then conducting isolation and purification using silica gel, gel, and C-18 reverse-phase column chromatography isolation techniques, to obtain the meroterpenoid I.

Specifically, the marine fungus Aspergillus terreus GZU-311 strain in S1 was deposited at the Guangdong Microbial Culture Collection Center on Dec. 17, 2019, with the accession number GDMCC No: 60789.

Specifically, a method for preparing the seed solution in the S1 includes: inoculating the marine fungus Aspergillus terreus GZU-311 into a slant medium for cultivation, and then inoculating same into a liquid medium for cultivation to obtain the seed solution, with a cultivation temperature being 28° C.-35° C., and a cultivation duration being 4-10 days.

More specifically, the cultivation temperature is 30° C., and the cultivation duration is 6 days.

Specifically, the step S2 involves inoculating the seed solution prepared in the step S1 into the solid maize medium for stationary cultivation to obtain the fermentation culture, with a cultivation temperature being 28° C.-35° C., and a cultivation duration being 30-60 days.

More specifically, the cultivation temperature is 30° C., and the cultivation duration is 60 days.

Specifically, a formulation of the slant medium in the step S1 is as follows: based on mass ratio, 0.3% glucose, 0.1% yeast extract, 0.1%-0.5% peptone, 1.5%-2.5% agar, 1.5%-4% sodium chloride, and water to make up 100%.

More specifically, the formulation of the slant medium in S1 is as follows: based on mass ratio, 0.3% glucose, 0.1% yeast extract, 0.5% peptone, 2.5% agar, 3% sodium chloride, and water to make up 100%.

Specifically, a formulation of the solid maize medium in the step S2 is as follows: based on mass ratio, maize:seawater=1:1 to 1:2.

More specifically, the formulation of the solid maize medium in the step S2 is as follows: based on mass ratio, maize:seawater=1:1.

Specifically, the number of times of methanol extraction in the step S3 is three.

The present invention provides an anti-heart failure drug, containing the meroterpenoid I of the present invention or a pharmaceutically acceptable salt thereof.

Specifically, the drug further includes the meroterpenoid I or a pharmaceutically acceptable salt thereof, and a pharmaceutically acceptable excipient.

Specifically, the meroterpenoid I may be administered alone or in the form of a pharmaceutical composition. The pharmaceutical composition can be formulated into various suitable pharmaceutical preparations according to the route of administration.

Optionally, the pharmaceutical preparation is an oral preparation, an injectable preparation, or a topical transdermal preparation.

Specifically, the oral preparation can be formulated into any orally acceptable preparation form, including, but not limited to, a tablet, a capsule, an aqueous solution, or an aqueous suspension. A carrier for use in the tablet generally includes lactose and maize starch; and additionally, a lubricant such as magnesium stearate may be added. A diluent used in the capsule preparation generally includes lactose and dried maize starch. The aqueous suspension is typically prepared by mixing an active ingredient with a suitable emulsifier and a suitable suspending agent.

Optionally, some sweeteners, flavoring agents, or coloring agents may also be added to the aforementioned oral preparation.

Specifically, the injectable preparation includes a liquid injection, a powder for injection, or a tablet for injection. The injectable preparation can be administered in the form of a sterile injectable preparation, including a sterile injectable aqueous or oily suspension or a sterile injectable solution. Optional carrier and solvent for use in the injectable preparation includes water, an isotonic sodium chloride solution such as Ringer's solution, and sterile non-volatile oils such as monoglycerides and diglycerides.

Specifically, the topical transdermal preparation includes a spray, a gel, an emulsion, an ointment, a lotion, a cream, or a gel patch. A carrier for the ointment includes, but is not limited to: mineral oil, liquid vaseline, white vaseline, propylene glycol, polyethylene oxide, polypropylene oxide, emulsifying wax, and water. A carrier for the lotion or cream includes, but is not limited to: mineral oil, sorbitan monostearate, Tween 60, cetyl ester wax, hexadecanol, 2-octyldodecanol, benzyl alcohol, and water.

Specifically, the pharmaceutically acceptable excipient includes one or more of a pharmaceutical carrier, a surfactant, a buffering substance, a disintegrant, a binder, a filler, a lubricant, a vehicle, a solubilizer, a flavoring agent, and a coloring agent.

Specifically, the pharmaceutical preparation can be administered in any of the following ways: orally, via spray inhalation, rectally, nasally, topically, or parenterally, wherein parenteral administration includes, for example, subcutaneous, intravenous, intramuscular, intraperitoneal, intrathecal, intraventricular, intrasternal, and/or intracranial injection or infusion, or administration by means of an implanted reservoir.

Specifically, the pharmaceutical preparation is preferably administered orally, intraperitoneally, or intravenously.

In addition, it needs to be noted that the use dose and method of the meroterpenoid I of the present invention depend on numerous factors, including the patient's age, body weight, gender, natural health status, nutritional status, the activity of the compound, the timing of administration, metabolic rate, the severity of the condition, and the subjective judgment of the attending physician.

The present invention has the following beneficial effects:

In the present invention, mouse heart failure models induced by doxorubicin or left anterior descending coronary artery ligation are treated with meroterpenoid I isolated and purified from marine fungus Aspergillus terreus GZU-311. It is found that the meroterpenoid I can improve cardiac function in mice, reduce cardiomyocyte injury, inhibit myocardial connective tissue thickening, ameliorate disordered arrangement of cardiomyocytes, and reduce inflammatory cell infiltration. Therefore, the present invention provides the use of meroterpenoid I in the preparation of an anti-heart failure drug. The present invention enriches pharmaceutical molecules available for the treatment of heart failure, thereby facilitating the development of anti-heart failure drugs and their clinical treatment, demonstrating promising prospects for clinical application.

### BRIEF DESCRIPTION OF THE DRAWINGS

FIG. 1 shows echocardiograms of a doxorubicin-induced mouse heart failure model and a mouse heart failure model treated with meroterpenoid I.

FIG. 2 shows results of cardiac function detection for a doxorubicin-induced mouse heart failure model and a mouse heart failure model treated with meroterpenoid I, where A shows computation results of ejection fraction (EF), and B shows computation results of fractional shortening (FS); #represents P<0.05; * represents P<0.05; ** represents P<0.01; *** represents P<0.001.

FIG. 3 shows pictures of heart section HE staining of a doxorubicin-induced mouse heart failure model and a mouse heart failure model treated with meroterpenoid I, with a proportional scale of 100 μm.

FIG. 4 shows pictures of heart section Masson staining of a doxorubicin-induced mouse heart failure model and a mouse heart failure model treated with meroterpenoid I, with a proportional scale of 50 μm.

FIG. 5 shows echocardiograms of a mouse heart failure model induced by left anterior descending coronary artery ligation and a mouse heart failure model treated with meroterpenoid I.

FIG. 6 shows results of cardiac function detection for a mouse heart failure model induced by left anterior descending coronary artery ligation and a mouse heart failure model treated with meroterpenoid I, where A shows computation results of ejection fraction (EF), and B shows computation results of fractional shortening (FS); ###represents P<0.001; *** represents P<0.001.

FIG. 7 shows pictures of heart section HE staining of a mouse heart failure model induced by left anterior descending coronary artery ligation and a mouse heart failure model treated with meroterpenoid I, with a proportional scale of 50 μm.

FIG. 8 shows results of blood pressures in an angiotensin II-induced mouse heart failure model and a mouse heart failure treated with meroterpenoid I; systolic blood pressure (SBP) and diastolic blood pressure (DBP); **** represents P<0.0001.

FIG. 9 shows echocardiograms of an angiotensin II-induced mouse heart failure model and a mouse heart failure model treated with meroterpenoid I.

FIG. 10 shows results of cardiac function detection for an angiotensin II-induced mouse heart failure model and a mouse heart failure model treated with meroterpenoid I; computation results of ejection fraction (EF) and fractional shortening (FS); where ###represents P<0.001; *** represents P<0.001.

FIG. 11 shows echocardiograms of a model of mice with heart failure with preserved ejection fraction and a mouse heart failure model treated with meroterpenoid I.

FIG. 12 shows results of cardiac function detection of a model of heart failure with preserved ejection fraction and a mouse heart failure model treated with meroterpenoid I, where A shows computation results of ejection fraction (EF), B shows the ratio of early to late diastolic mitral inflow peak velocity (E/A), and C shows the ratio of early diastolic mitral inflow peak velocity to early diastolic mitral annular peak velocity (E/e′); ###represents P<0.001; *** represents P<0.001; **** represents P<0.0001.

FIG. 13 shows results of blood pressures in a model of heart failure with preserved ejection fraction and a mouse heart failure treated with meroterpenoid I, where A shows computation results of systolic blood pressure (SBP), and B shows computation results of diastolic blood pressure (DBP); **** represents P<0.0001.

### DESCRIPTION OF THE EMBODIMENTS

The present invention is further illustrated below in conjunction with the drawings of the specification and specific embodiments, but the embodiments do not limit the present invention in any form. The reagents, methods, and devices employed in the present invention are those conventional in the technical field, unless otherwise specified.

The reagents and materials used in the following examples are all commercially available, unless otherwise specified.

### Example 1 Obtaining of Meroterpenoid I from Marine Fungus

The meroterpenoid I in the present invention was isolated and purified from the fermentation culture of marine fungus Aspergillus terreus GZU-311. The marine fungus Aspergillus terreus GZU-311 was deposited at the Guangdong Microbial Culture Collection Center on Dec. 17, 2019, with the accession number GDMCC No: 60789. The deposit address is the Guangdong Institute of Microbiology, 5th Floor, Building 59, Compound No. 100, Xianlie Middle Road, Guangzhou. Relevant isolation and identification information has been disclosed in the patent application with the publication No. CN111139188A.

The isolation, purification, and identification process for the meroterpenoid I was as follows:

### 1. Isolation of COMPOUND

The method for isolating the compound included the following steps:
(1) the marine fungus Aspergillus terreus GZU-311 was inoculated onto a slant medium (the formulation of the slant medium: based on mass ratio, 0.3% glucose, 0.1% yeast extract, 0.5% peptone, 2.5% agar, 3% sodium chloride, and water to make up 100%); subsequently, it was inoculated into a liquid medium (having the same formulation as the slant medium, with the exception of containing no agar) and it was cultivated at 30° C. for 6 days to obtain a seed solution;
(2) the seed solution was inoculated into a solid maize medium (the formulation of the solid maize medium: based on mass ratio, maize:seawater=1:1) and was standing at 30° C. for 60 days to obtain a fermentation culture (fermentation broth);
(3) the fermentation culture was extracted three times with methanol, and the extracts were concentrated to a paste; the resulting concentrated extract was extracted with ethyl acetate to obtain a crude ethyl acetate extract;
(4) the crude ethyl acetate extract was isolated using normal-phase silica gel chromatography, and then subjected to gradient elution in sequence using petroleum ether/ethyl acetate mixed solutions with petroleum ether volume fractions of 10%, 20%, 30%, 40%, 50%, 60%, and 70%, respectively. The 40% petroleum ether/ethyl acetate eluate fraction was collected and then subjected to isolation and purification in sequence using silica gel, gel, and C-18 reverse-phase column chromatography isolation techniques. The compound obtained from the isolation and purification was identified.

### 2. Identification of Compound

The compound obtained from the isolation and purification was subjected to structural test analysis using high-resolution mass spectrometry and nuclear magnetic resonance (NMR), yielding the following experimental data:

The molecular formula of the compound: C₂₆H₃₂O₉, high-resolution mass spectrometry (HRESI-MS): 487.1959 [M-H]⁻ (cald for C₂₆H₃₁O₉ 487.19626).

The structural formula of the compound is shown below (formula (I)):

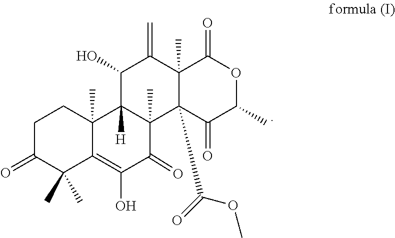

The NMR data for the compound are shown in Table 1.

TABLE 1
NMR Data (CDCl₃, 400 MHz/100 MHz, ppm)

| No | δ_{H} (J in Hz) | δ_{C} |
|---|---|---|
| 1 | 2.64, m | 32.5, CH₂ |
| 2 | 2.32, dd (13.0, 8.4) | 34.3, CH₂ |
| | 1.82, m | |
| 3 | | 214.3, C |
| 4 | | 48.0, C |
| 5 | | 138.3, C |
| 6 | | 139.1, C |
| 7 | | 196.1, C |
| 8 | | 49.2, C |
| 9 | 1.96, d (2.3) | 48.5, CH |
| 10 | | 39.2, C |
| 11 | 4.85, m | 73.3, CH |
| 12 | | 147.0, C |
| 13 | | 54.1, C |
| 14 | | 66.8, C |
| 15 | | 204.5, C |
| 16 | 4.65, q (6.7) | 78.8, CH |
| 17 | | 170.3, C |
| 18 | 1.53, s | 24.6, CH₃ |
| 19 | 1.50, s | 20.9, CH₃ |
| 20 | 2.12, s | 20.5, CH₃ |
| 21 | 1.37, s | 20.2, CH₃ |
| 22 | 5.09, s; 5.29, s | 117.3, CH₃ |
| 23 | 2.11, s | 23.5, CH₃ |
| 24 | 1.55, d (6.7) | 18.3, CH₃ |
| 25 | | 167.0, CH₃ |
| 26 | 3.82, s | 53.3, CH₃ |
| 6-OH | 6.54, s | |
| 11-OH | 1.76, s | |

Upon identification, the compound obtained from the isolation and purification was a meroterpenoid and was designated as meroterpenoid I.

### Example 2 Effect of Meroterpenoid I in Treatment of Doxorubicin-Induced Heart Failure

### 1. Experimental Method

8-week-old male C57BL/6 mice were randomly equally divided into six groups (8 mice per group): Control Group, Model Group, Low-Dose Meroterpenoid I Group (I (10 mg/kg)), Medium-Dose Meroterpenoid I Group (I (20 mg/kg)), High-Dose Meroterpenoid I Group (I (40 mg/kg)), and Positive Drug Sacubitril Valsartan Group (XST).

The daily intragastric administration doses were as follows: the meroterpenoid I (dissolved in 10% DMSO+normal saline) was administered at doses of 10, 20, and 40 mg/kg, respectively; the positive drug XST was administered at 40 mg/kg; and the Control Group and the Model Group received an equal volume of the blank solvent (10% DMSO+normal saline). Meanwhile, doxorubicin was administered via subcutaneous injection once a week at a single dose of 5 mg/kg, while the control group was injected with an equal volume of normal saline. The specific dose for intragastric administration was set according to the mouse's body weight. For example, “10 mg/kg” indicated that a mouse with a body weight of 1 kg received a dose of 10 mg.

On the 29th day following intragastric administration, the cardiac structure and function of mice in each group were assessed using a VINNO 6 small-animal high-resolution micro-ultrasound imaging system, and the ejection fraction (EF) and the fractional shortening (FS) were calculated. Subsequently, the mice were dissected to harvest their hearts, which were then embedded, sectioned, and subjected to HE and Masson staining.

### 2. Data Processing

The experimental results were statistically analyzed using GraphPad Prism 8.0 software and expressed as Mean±SEM. Inter-group comparisons were performed using One-way ANOVA.

### 3. Experimental Result

### (1) Assessment of Cardiac Function

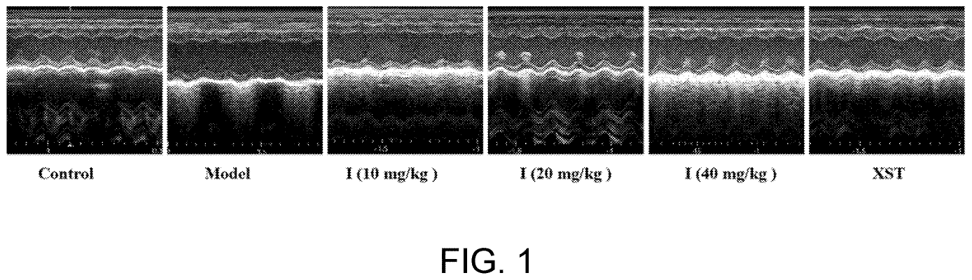

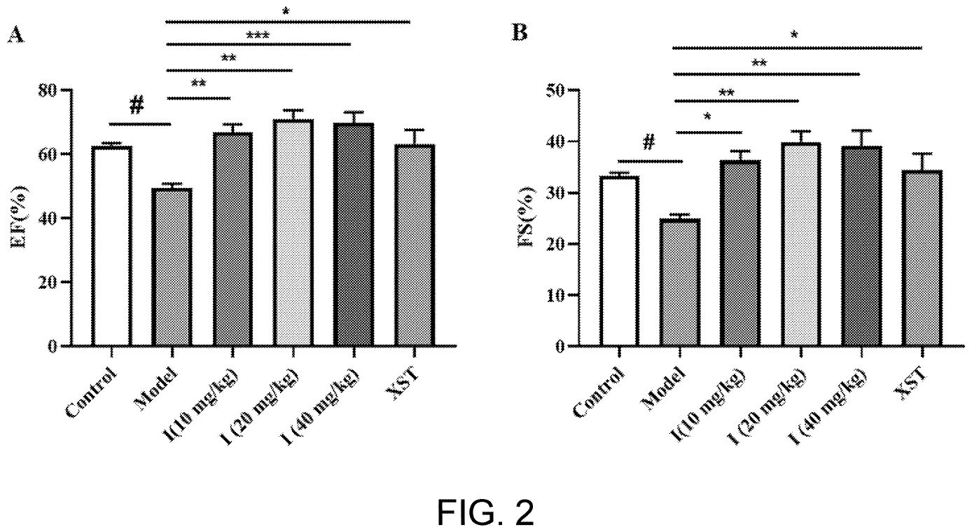

The echocardiograms of the doxorubicin-induced mouse heart failure model and the mouse heart failure model treated with the meroterpenoid I are shown in FIG. 1. The results of cardiac function detection for the doxorubicin-induced mouse heart failure model and the mouse heart failure model treated with the meroterpenoid I are shown in FIG. 2. As can be seen from FIG. 1 and FIG. 2, compared with the Control Group, mice in the Model Group exhibited a significant decrease in cardiac ejection fraction (EF) and fractional shortening (FS), as well as a significant increase in left ventricular end-systolic and end-diastolic volumes; that is, the cardiac function in the doxorubicin-induced mice was significantly reduced. Compared with the Model Group, mice treated with the meroterpenoid I exhibited a significant increase in cardiac ejection fraction and fractional shortening, as well as a significant decrease in left ventricular end-systolic and end-diastolic volumes, such that the cardiac function in the doxorubicin-induced mice can be significantly improved.

### (2) Heart Section HE Staining

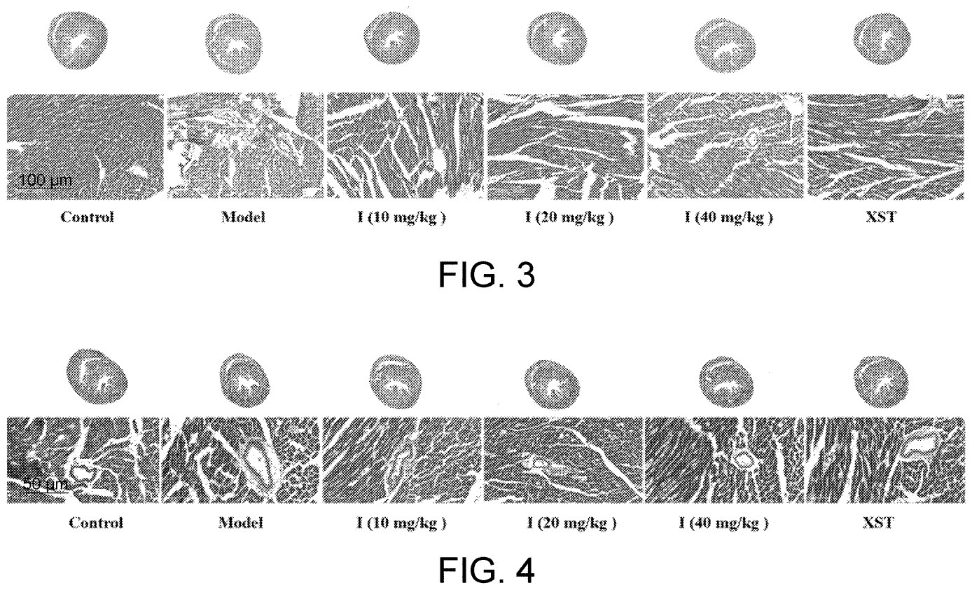

The pictures of heart section HE staining of the doxorubicin-induced mouse heart failure model and the mouse heart failure model treated with the meroterpenoid I are shown in FIG. 3. As can be seen from FIG. 3, compared with the Control Group, the cardiomyocytes of mice in the Model Group exhibited significant injury, while heart sections of mice treated with the meroterpenoid I at a dose of 40 mg/kg exhibited little or no cardiomyocyte injury, with cells arranged regularly and densely. This indicates that the meroterpenoid I can reduce cardiomyocyte injury and exerts a certain protective effect on cardiac tissue.

### (3) Heart Section Masson Staining

The pictures of heart section Masson staining of the doxorubicin-induced mouse heart failure model and the mouse heart failure model treated with the meroterpenoid I are shown in FIG. 4. As can be seen from FIG. 4, the Control Group exhibited only sparse regions of connective tissue thickening, while the sections from the Model Group exhibited distinct extensive regions of connective tissue thickening; compared with the Model Group, treatment with the meroterpenoid I at a dose of 40 mg/kg can significantly reduce doxorubicin-induced connective tissue thickening.

### Example 3 Effect of Meroterpenoid I Against Ischemic Heart Failure Induced by Left Anterior Descending Coronary Artery Ligation

### 1. Experimental Method

### (1) Experimental Materials

8-week-old male C57BL/6 mice, purchased from Guangdong Zhiyuan Biomedical Technology Co., Ltd., were fed a standard diet with free access to food and water.

### (2) Experimental Modeling

Modeling was performed using in situ ligation of the left anterior descending (LAD) coronary artery via thoracotomy. First, mice were anesthetized via intraperitoneal injection of 0.25% sodium pentobarbital, and a ventilator intubation tube was inserted. Ventilator parameters were set as follows: tidal volume of 11 cc, inspiratory-to-expiratory ratio of 1:2, and respiratory rate of 144 breaths/min. The incision was covered with normal saline gauze. A 1 cm incision was made in the intercostal space between the 3rd and 4th ribs, to the left of the sternum. Two spreaders were utilized to spread the ribs in coordination in the up, down, left, and right directions to expose the heart. The pericardium was opened. Using a modern standard code 6-0 silk suture and a round needle, the needle was inserted 3-4 mm below the left atrial appendage at the bifurcation of the left coronary artery and the posterior descending artery, to a depth of approximately 1 mm and a width of approximately 2 mm. After inserting the needle and passing the suture, the ligature was tightened. The electrocardiogram was observed immediately; once ST-segment elevation or widened QRS complexes were observed, the incision was sutured to close the chest. Four weeks after postoperative feeding, a small-animal color ultrasound was performed to detect the cardiac function, with an EF of less than 45% considered indicative of successful modeling.

### (3) Experimental Grouping and Data Measurement

The experimental mice were divided into the following four groups (5 mice per group): Blank Control Group (thoracotomy followed by suturing, without coronary artery ligation; Sham), Model Group (CHF), Model Group+Meroterpenoid I Group (I (20 mg/kg)), and Model Group+Sacubitril Valsartan Group (XST).

The intragastric administration doses were as follows: the Model Group+Meroterpenoid I Group (dissolved in 10% DMSO+normal saline) was administered at 20 mg/kg; the Model Group+Sacubitril Valsartan Group was administered at 26 mg/kg; and the Blank Control Group and the Model Group received an equal volume of the solvent (10% DMSO+normal saline). The specific dose for intragastric administration was set according to the mouse's body weight. For example, “20 mg/kg” indicated that a mouse with a body weight of 1 kg received a dose of 20 mg. Intragastric administration was performed once daily for a total of 4 weeks.

On the 29th day following intragastric administration, the cardiac structure and function of mice in each group were assessed using a VINNO 6 small-animal high-resolution micro-ultrasound imaging system, and the ejection fraction (EF) and the fractional shortening (FS) were calculated. Subsequently, the mice were dissected to harvest their hearts, which were then embedded, sectioned, and subjected to HE staining.

### 2. Data Processing

The experimental data were statistically analyzed using GraphPad Prism 8.0 software, and the experimental results were expressed as Mean±SEM. Inter-group comparisons were performed using One-way ANOVA.

### 3. Experimental Result

### (1) Assessment of Cardiac Function

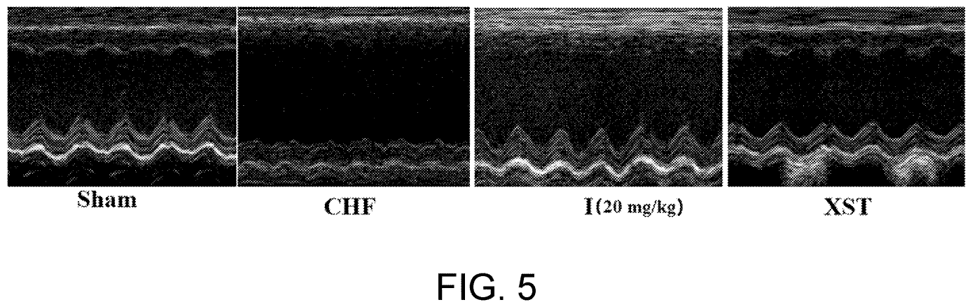

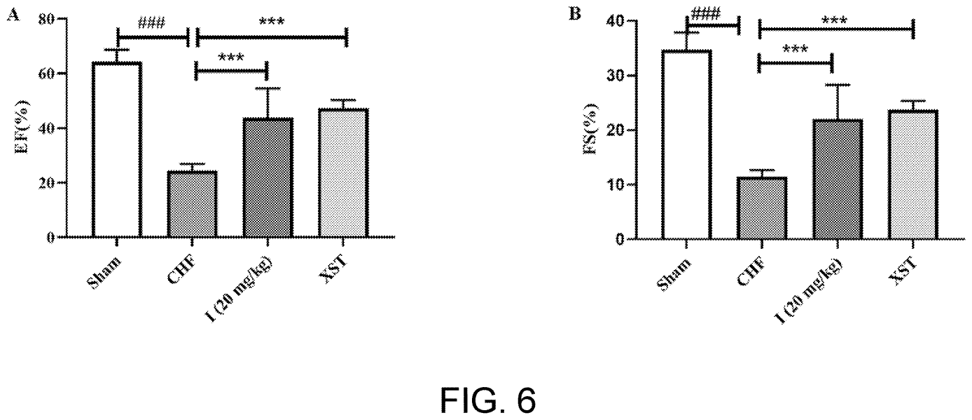

The echocardiograms of the mouse heart failure model induced by left anterior descending coronary artery ligation and the mouse heart failure model treated with the meroterpenoid I are shown in FIG. 5. The results of cardiac function detection for the mouse heart failure model induced by left anterior descending coronary artery ligation and the mouse heart failure model treated with the meroterpenoid I are shown in FIG. 6. As can be seen from FIG. 6, compared with the Blank Control Group, mice in the Model Group exhibited a significant decrease in cardiac ejection fraction (EF) and fractional shortening (FS), indicating a significant reduction in cardiac function of mice in the Model Group; compared with the Model Group, treatment with the meroterpenoid I at a dose of 20 mg/kg can significantly increase the EF and FS values in the heart failure model induced by left anterior descending coronary artery ligation, thereby improving the cardiac function in the mice with heart failure.

### (2) Heart Section HE Staining

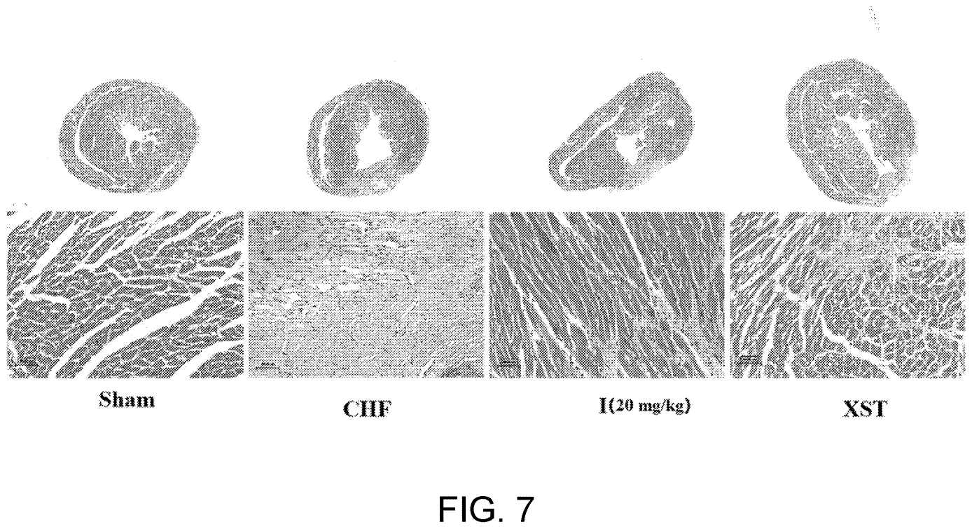

The pictures of heart section HE staining of the mouse heart failure model induced by left anterior descending coronary artery ligation and the mouse heart failure model treated with the meroterpenoid I are shown in FIG. 7. As can be seen from FIG. 7, compared with the Control Group, the cardiomyocytes in the Model Group exhibited disordered arrangement and extensive infiltration by inflammatory cells. Compared with the Model Group, treatment with the meroterpenoid I at a dose of 20 mg/kg can significantly reduce inflammatory cell infiltration and maintain the arrangement of cardiomyocytes.

### Example 4 Effect of Meroterpenoid I in Treatment of Angiotensin II-Induced Heart Failure

### 1. Experimental Method

6-8-week-old male C57BL/6J mice were randomly allocated: 6 mice were for the Control Group and treated with PBS; the remaining mice were treated with Ang II to establish a chronic heart failure model. Ang II was dissolved in sterile PBS containing 0.01 mol/L acetic acid. Under sterile conditions, the Ang II solution was carefully loaded into an Alzet osmotic pump, avoiding the formation of air bubbles. The osmotic pump was immersed in normal saline and placed in a 37° C. incubator for 48 hours to activate its osmotic system (flow rate set to 1.5 ug/min/kg). After each mouse was subjected to gaseous anesthesia with isoflurane, an incision of approximately 1 cm was made in the posterior dorsal region of the neck, and the osmotic pump was implanted subcutaneously. Osmotic pumps containing PBS were implanted into mice in the Control Group. Postoperatively, each mouse was injected intraperitoneally with 600 units of penicillin for three consecutive days to prevent infection. Blood pressure measurement was performed two weeks after modeling. Mice with successful modeling were divided into the following groups, with 6 mice per group: Model Group: continued Ang II intervention. Positive Drug Group: received Sacubitril Valsartan Sodium tablets (LCZ696) at a dose of 30 mg/kg. Low-Dose Meroterpenoid I Group: received meroterpenoid I at a dose of 2.5 mg/kg. Middle-Dose Meroterpenoid I Group: received meroterpenoid I at a dose of 5 g/kg. High-Dose Meroterpenoid I Group: received meroterpenoid I at a dose of 10 mg/kg. Control Group, namely mice treated only with PBS, was designated as control group. Two weeks after modeling, with the exception of the control group, which received a daily, timed intragastric administration of 100 μL of normal saline, the remaining administration groups underwent corresponding intragastric drug administration according to their respective grouping, once daily. Administration was performed for four weeks, and blood pressure level examination was performed once a week from the beginning to the end. At the end of the experiment, cardiac ultrasound and blood pressure examination were performed, and blood and heart samples of mice were collected. The samples were stored in a −80° C. freezer. Sacubitril valsartan was dissolved in ddH₂O, and APF was first dissolved in DMSO and then diluted with maize oil. For example, “10 mg/kg” indicated that a mouse with a body weight of 1 kg received a dose of 10 mg.

Four weeks after intragastric administration, the cardiac structure and function of mice in each group were assessed using a VINNO 6 small-animal high-resolution micro-ultrasound imaging system, and the ejection fraction (EF) and the fractional shortening (FS) were calculated.

### 2. Data Processing

The experimental results were statistically analyzed using GraphPad Prism 8.0 software and expressed as Mean±SEM. Inter-group comparisons were performed using One-way ANOVA.

### 3. Experimental Result

### (1) Blood Pressure Assessment

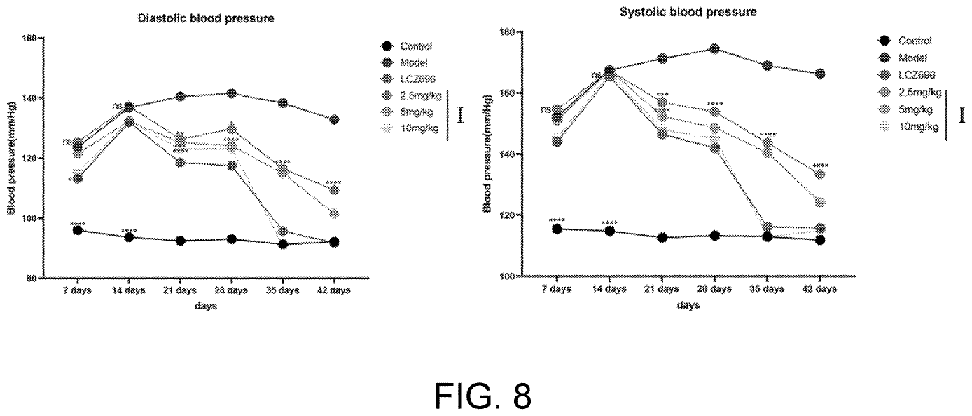

The systolic and diastolic blood pressures in the angiotensin II-induced mouse heart failure model and the mice treated with the meroterpenoid I are shown in FIG. 8. As can be seen from FIG. 8, compared with the Blank Control Group, mice in the Model Group exhibited a significant increase in systolic and diastolic blood pressures; compared with the Model Group, treatment with the meroterpenoid I can significantly reduce the systolic and diastolic blood pressures in mice.

### (2) Assessment of Cardiac Function

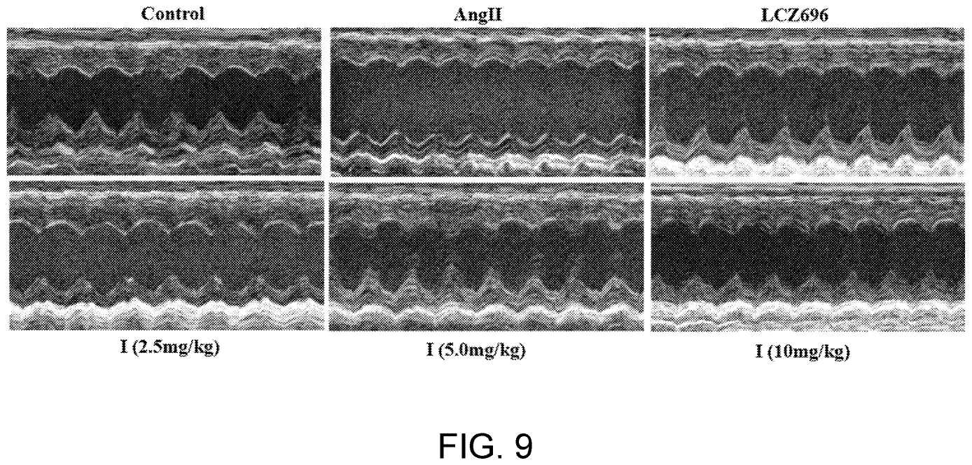

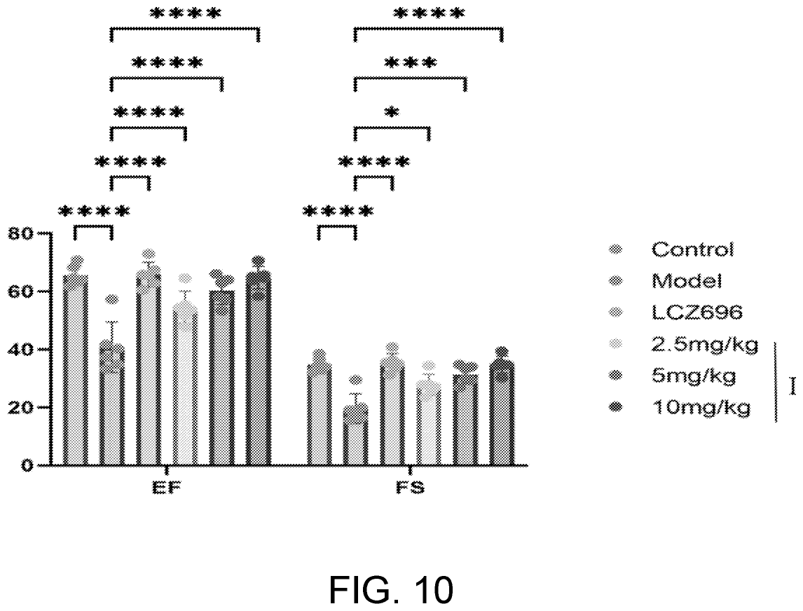

The echocardiograms of the angiotensin II-induced mouse heart failure model and the mouse heart failure model treated with the meroterpenoid I are shown in FIG. 9. The results of cardiac function detection for the angiotensin II-induced mouse heart failure model and the mouse heart failure model treated with the meroterpenoid I are shown in FIG. 10. As can be seen from FIG. 9 and FIG. 10, compared with the Control Group, mice in the Model Group exhibited a significant decrease in cardiac ejection fraction (EF) and fractional shortening (FS), that is, the cardiac function in the angiotensin II-induced mice was significantly reduced. Compared with the Model Group, mice treated with the meroterpenoid I exhibited a significant increase in cardiac ejection fraction and fractional shortening, such that the cardiac function in the angiotensin II-induced mice can be significantly improved.

### Example 5 Effect of Meroterpenoid I in Treatment of Heart Failure with Preserved Ejection Fraction

### 1. Experimental Method

After one week of acclimatization, 6-8-week-old female C57BL/6J mice were randomly divided into six groups: Blank Control Group, Model Control Group, Positive Control Group (empagliflozin), Low-Dose Meroterpenoid I Group (2.5 mg/kg), Medium-Dose Meroterpenoid I Group (5.0 mg/kg), and High-Dose Meroterpenoid I Group (10 mg/kg), with 8 mice per group. With the exception of the Blank Control Group, which was fed normal maintenance feed and provided with purified drinking water, mice in all other groups were subjected to a high-fat diet combined with L-NAME administration via drinking water to establish a model of heart failure with preserved ejection fraction. L-NAME was dissolved in drinking water at a final concentration of 0.5 g/L, and at the same time, the mice were fed a 60% high-fat feed for 8 consecutive weeks.

Following successful modeling, intragastric administration was initiated for each administration group. The meroterpenoid I was first dissolved in DMSO and then diluted with maize oil to the corresponding concentration; the positive control drug, empagliflozin, was similarly dissolved in DMSO and then diluted with maize oil. The Blank Control Group and the Model Control Group received an equal volume of normal saline via intragastric administration. The administration volume was 0.1 mL/10 g, the administration frequency was once daily, and administration was continued for 4 weeks. During the administration, the administration dose was adjusted weekly according to the changes in body weight.

Four weeks after intragastric administration, various indices were detected: the cardiac structure and function of mice in each group were assessed using a small-animal ultrasound imaging system. After being anesthetized via isoflurane inhalation, the mice were placed in a supine position and secured to a heating plate at a constant temperature of 37° C. The M mode was employed to measure the left ventricular systolic function and calculate the ejection fraction; pulse Doppler and tissue Doppler were employed to measure the ratio of early to late diastolic mitral inflow peak velocity (E/A), as well as the ratio of early diastolic mitral inflow peak velocity to early diastolic mitral annular peak velocity (E/e′), in order to assess the cardiac diastolic function. All of the above parameters were measured three times, and the average value was taken. The systolic and diastolic blood pressure of mice were detected using a non-invasive tail artery blood pressure measuring instrument. The mice were placed in a constant-temperature holder; after dilation of the tail artery, measurements were performed continuously more than five times while the mice remained in a quiescent state. The average value was calculated after the maximum and minimum values were excluded.

### 2. Data Processing

The experimental results were statistically analyzed using GraphPad Prism 8.0 software, and all data were expressed as Mean±SEM. When the data satisfied the normal distribution and homogeneity of variances, inter-group comparisons were performed using One-way ANOVA.

### 3. Experimental Result

### (1) Assessment of Cardiac Function

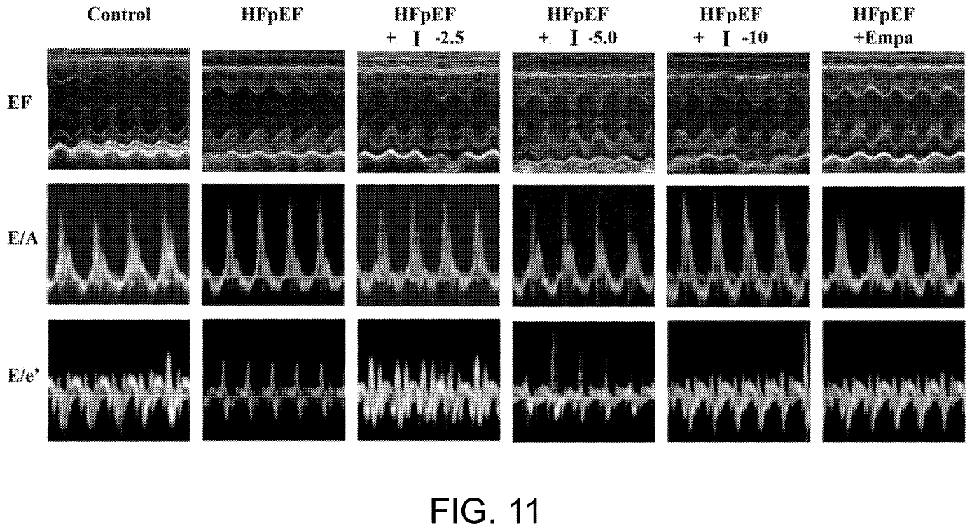

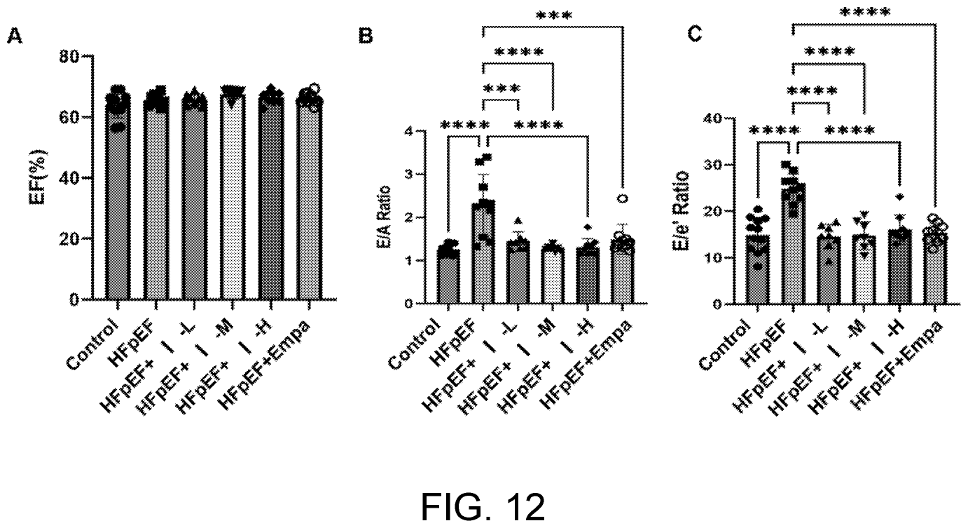

The echocardiograms of the model of heart failure with preserved ejection fraction and the mice treated with the meroterpenoid I are shown in FIG. 11; the results of cardiac function detection are shown in FIG. 12. Compared with the Blank Control Group, mice in the Model Control Group exhibited no significant changes in cardiac ejection fraction and left ventricular fractional shortening; however, the E/A ratio reflecting the diastolic function was significantly decreased, and the E/e′ ratio was significantly increased, suggesting the presence of diastolic dysfunction in mice in the Model Group, such that the model of heart failure with preserved ejection fraction was established successfully. Compared with the Model Control Group, mice treated with the meroterpenoid I exhibited a significant increase in the E/A ratio and a significant decrease in the E/e′ ratio, indicating that the meroterpenoid I can significantly improve the diastolic function in mice with heart failure with preserved ejection fraction.

### (2) Blood Pressure Assessment

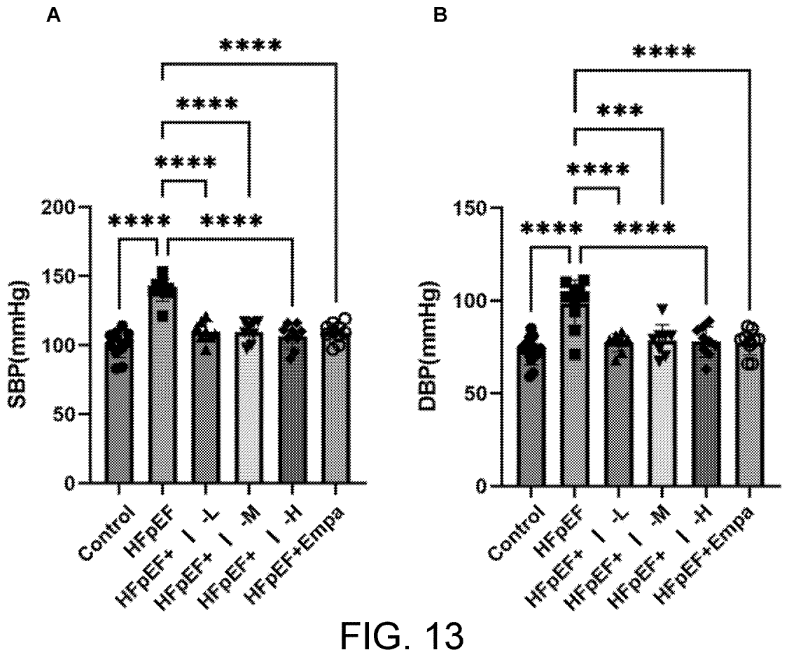

The systolic and diastolic blood pressures of the model of heart failure with preserved ejection fraction and the mice treated with the meroterpenoid I are shown in FIG. 13. Compared with the Blank Control Group, mice in the Model Control Group exhibited a significant increase in systolic and diastolic blood pressures. Compared with the Model Control Group, treatment with the meroterpenoid I can dose-dependently reduce both the systolic and diastolic blood pressure in mice.

The aforementioned results indicate that the meroterpenoid I of the present invention has an anti-heart failure effect, and it can be applied in the preparation of anti-heart failure drugs, demonstrating promising prospects for clinical application.

The aforementioned embodiments represent preferred implementations of the present invention. However, the implementations of the present invention are not limited to these embodiments. Any other alterations, modifications, substitutions, combinations, or simplifications made without departing from the spirit and principle of the present invention shall be regarded as equivalent replacement methods and are encompassed within the scope of protection of the present invention.

What is claimed is:

1. A method of preparing an anti-heart failure drug, comprising a step of using meroterpenoid I, wherein a structural formula of the meroterpenoid I is shown as formula (I):

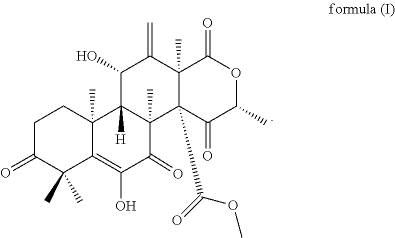

2. The method according to claim 1, wherein the meroterpenoid I is isolated and purified from a fermentation culture of marine fungus Aspergillus terreus GZU-311; and a medium used for fermentation cultivation is a medium formulated from maize and seawater.

3. The method according to claim 1, wherein the meroterpenoid I is capable of improving cardiac function.

4. The method according to claim 3, wherein the meroterpenoid I is capable of increasing a cardiac ejection fraction and a fractional shortening.

5. The method according to claim 1, wherein the meroterpenoid I is capable of reducing cardiomyocyte injury.

6. The method according to claim 1, wherein the meroterpenoid I is capable of inhibiting myocardial connective tissue thickening.

7. The method according to claim 1, wherein the meroterpenoid I is capable of ameliorating disordered arrangement of cardiomyocytes.

8. The method according to claim 1, wherein the meroterpenoid I is capable of reducing inflammatory cell infiltration.

9. An anti-heart failure drug, comprising the meroterpenoid I in claim 1 or a pharmaceutically acceptable salt thereof.

10. The drug according to claim 9, further comprising a pharmaceutically acceptable excipient.
